(19) United States
(12) Patent Application Publication
(10) Pub. No.: US 2017/0081693 A1
(43) Pub. Date: Mar. 23, 2017

(54) PROCESS FOR PREPARING CAROTENOIDS BY SUBMERGED FERMENTATION WITH MIXED CULTURES OF (+) AND (-) STRAINS OF THE FUNGUS BLAKESLEA TRISPORA

(71) Applicant: HOCHSCHULE ANHALT, Köthen (DE)

(72) Inventors: Robert Reinhard PÄTZ, Köthen (DE); Thomas PAPERT, Dessau-Rosslau (DE); Vivien PETER, Köthen (DE); Sarah POLAGE, Leisnig OT Görnitz (DE)

(21) Appl. No.: 15/310,547

(22) PCT Filed: May 12, 2015
(86) PCT No.: PCT/EP2015/060466

§ 371 Date: Nov. 11, 2016

(30) Foreign Application Priority Data
May 12, 2014 (DE) 10 2014 208 876.3

(51) Int. Cl. C12P 23/00; A61K 31/01; A23D 7/005; A61K 31/015

(52) U.S. Cl. CPC C12P 23/00; A61K 31/015; A61K 31/01; A23D 7/005

(57) ABSTRACT

The invention concerns a simple and effective process for preparing β-carotene or lycopene by submerged fermentation with mixed cultures of (+) and (−) strains of the fungus Blakeslee trispora, characterized by high productivity of the B. trispora strains. The high productivity is achieved by virtue of the inventive regime, which acknowledges both the morphological state of the hemi-strains with their filamentous growth state when the strains are mixed (referred to as mating).

The invention relates to a simple and effective method for producing β-carotene or lycopene through submerged fermentation with mixed cultures of (+) and (−) strains of the fungus Blakeslea trispora, which is characterized by high productivity of the B. trispora strains. The high productivity is achieved by virtue of the inventive regime, which acknowledges both the morphological state of the filamentous growing hemi-strains and the growth state when the strains are mixed (referred to as mating).

Carotenoids are natural dyes, which create a yellow to red colour and which are frequently found in vegetable plants. They were and still are the subject of numerous studies due to their properties as antioxidants and as a precursor for vitamin A. Due to their antioxidant effect, they are said to prevent many diseases, such as cancer, arteriosclerosis, rheumatism or Alzheimer's. Lycopene (which is prevalent e.g. in tomatoes) here has the greatest antioxidative potential, and is regarded as providing effective protection against the particularly reactive singlet oxygen. On an industrial basis, carotenoids are used as food additives and dyeing agents, e.g. in margarines, oils, sauces, soups and fruit juices, but also as animal feed additives.

Carotenoids can be produced using chemical and biotechnology methods. When produced using biotechnology, carotenoids have a more complex structure, and the naturally prevalent conformational isomers are also very easy to access. The industrial biotechnology process for producing β-carotene is based on the use of the alga Dunaliella salina or the fungus Blakeslea trispora, wherein when B. trispora is used, a mixed fermentation of the (+) and (−) strains is conducted in order to obtain a maximum yield of β-carotene.

In the prior art, the production of β-carotene through fermentation of B. trispora is described in numerous patent documents, such as EP 1 367 131 A1, WO 93/20183 A1 or WO 02/010429 A1. They all describe fermentation methods based on the principle that the (+) and (−) hemi-strains are inoculated together in the bioreactor. WO 93/20183 A1 describes the production of β-carotene using selected mutated B. trispora strains under specific fermentation conditions. WO 02/010429 A1 discloses a fermentation method with a defined pH value regulation during fermentation, and the addition of soya lecithin as a culture medium in order to increase the generation of β-carotene in relation to other carotenoids.

The fermentation conditions described in EP 1 367 131 A1 provide for both a programmed addition of oxygen and/or beta-ions as well as a check of the vegetative growth phase of the strains used. Further fermentation methods are disclosed in U.S. Pat. No. 5,422,247 A and WO 2005/030976 A2, which comprise a regulation of the pH value and, if necessary, of the oxygen partial pressure.

In the literature, the use of chemical components is also described to increase production. Such ingredients can be toxic, however, and their legal authorisation for use as a foodstuff or for pharmaceutical purposes may be questionable.

The task of the present invention is therefore to provide an effective method for producing carotenoids, in particular β-carotene and lycopene, which provides good product yields without requiring chemical additives. Additionally, it should be as low-cost as possible, simple to implement and involve little complexity, and allow a reduction in the duration of fermentation.

The object of the invention is attained by means of a method for producing a carotenoid selected from β-carotene or lycopene through submerged fermentation with mixed cultures of (+) and (−) strains of the fungus Blakeslee trispora and extracting the carotenoid from the biomass obtained or from the oil phase of the fermentation sludge, which is characterized by the fact that, initially, the (−) and the (+) cell is pre-cultivated separately through to the full morphological formation, then the (−) cell is inoculated in the bioreactor, and during the exponential growth of the (−) strain, the (+) strain is added to the (−) strain in a volume ratio of 1:5 to 1:100 (also known as the mating ratio) for the induction of the carotenoid formation, and both strains are jointly fermented without pH regulation and without regulation of the oxygen partial pressure at 18 to 24° C., if necessary until the end of the carotenoid formation (idiophase).

The separated pre-cultivation of the strains is conducted in a preferred embodiment of the invention at 26-30° C. respectively, in a particularly preferred manner at 28° C. The separated pre-cultivation of the (−) strain is conducted for approx. 55 hours. After approx. 55 hours, the (−) strain is then transferred to the bioreactor. The fermentation of the (−) strain in the bioreactor is also preferably conducted at 26-30° C., in a particularly preferred manner at 28° C. The cultivation of the (−) strain in the bioreactor is conducted for approx. 13 to 14 hours (trophophase).

It is also particularly preferred that the joint fermentation of the (+) and (−) strain (idiophase) for carotenoid formation is conducted at 22° C.

According to the present invention, a cultivation of the (−) hemi-strain is conducted in the reactor scale up to a complete hyphen formation (trophophase), and only then, the (+) hemi-strain is added as an induction measure during the exponential growth of the (−) strain. As a result, the fermentation process can be shortened in terms of time, and costs can be saved. Additionally, a temperature shift occurs between the tropho- and idiophase from the 26-30° C. which is optimal for growth to 18 to 24° C. The lower temperature does not inhibit the vitality of the fungus hemi-strains, but does promote the stability of the product during fermentation. For successful cultivation with good product yields, no regulation of the pH or oxygen content is required in the present invention. Since no regulation of the pH value must be conducted, no use of acids or alkalis is necessary, and costs are saved.

The fumigation rate, rotation speed and temperature are kept constant during the entire product formation phase (idiophase), which clearly benefits an adaptation of the fungi to the prevalent conditions. The use of product formation is characterized by a similar reduction in the BDS (biological dry substance) content and pH value. The stop criterion for the carotenoid formation can be detected by a clear increase in the pH value. In other words, after a rise in pH value by 0.5 to 0.8, carotenoid formation is terminated.

The stirring speed during the separated pre-cultivation in the shaking flask of the strains is preferably 130 to 156 rpm.

It is preferred according to the invention that the stirring speed is set during cultivation of the (−) strain in the bioreactor (trophophase) to 230 to 350 rpm, particularly preferred to 244 to 344 rpm.

The fumigation volume flow during the trophophase is preferably approx. 1 vvm (volume air/volume fermenter/minute).

In the idiophase, i.e. in the phase of product formation, also known as the phase of joint fermentation of the (+) and (−) strains following induction, the conditions for the stirring speed and fumigation volume flow are the same as in the trophophase.

The mating ratio, given as the volume ratio (in each case (+) strain to (−) strain) is in a particular embodiment of the invention 1:5 to 1:20, particularly preferred 1:5 to 1:10.

In the idiophase of the method of the present invention, i.e. in the phase of product formation, also known as the phase of joint fermentation of the (+) and (−) strains following induction, the substrates generally known for B. trispora can be used as a substrate or fermentation substrate, in particular those which contain hydrocarbons and organic nitrogen compounds at the same time. Lipids can also be added to the substrate. According to the invention, starches or sugars are preferably used as hydrocarbons in the substrate. It has been shown that good yields of carotenoid are obtained with substrates containing sugar in particular, with a sugar content of 10 to 50 g/L. Substrates containing sugar preferably comprise molasses, beer wort or auxiliary products of the starch industry, or combinations of these. Preferably, the substrate contains lipids for the phase of joint fermentation of the (+) and (−) strains following induction. The lipids are preferably one or more oils. The oil(s) are preferably one or more native oils. In particular, vegetable oils, preferably native vegetable oils, can be used in the substrate. Examples of suitable vegetable oils are olive oil, rapeseed oil, maize germ oil and sunflower oil.

If lycopene is to be produced with the method according to the invention, it is preferred that during joint fermentation of the (+) and (−) strain (idiophase), a cyclase inhibitor is added to the substrate, preferably imidazole and/or an imidazole derivative. The method according to the invention also permits the production of lycopene without the addition of possibly toxic chemicals such as cyclase inhibitors. Here, lycopene is preferably extracted from the oil phase of the fermentation sludge.

In the method according to the invention, all known and publicly accessible B. trispora hemi-strains in culture collections, e.g. the (−) strain DSM 2388 and the (+) strain DSM 2387, or the (−) strain ATC 14272 and the (+) strain ATCC 14271. In one embodiment of the invention, the strain BS 01(−), which is stored in the international depository of the German Collection of Microorganisms and Cell Cultures GmbH (DSMZ) in Braunschweig, Germany, under the number DSM 16755, and the strain BS 01(+), which is also deposited there under the number DSM 16754, can be used.

In a very particular embodiment of the invention, the method is characterized by the following steps:
(1) The (−) strain is pre-cultivated separately for approx. 55 h and the (+) strain is pre-cultivated separately for approx. 68 h in the erlenmeyer flask at 28° C. and 145 rpm.
(2) After approx. 55 h, the (−) strain is transferred to a reactor and fermented for approx. 13-14 h at 28° C., a fumigation rate of approx. 1 vvm and a stirrer speed of 244 to 344 rpm.
(3) Following addition of the (+) strain, the cultivation temperature is set to 22° C. The volume ratio between (+) and (−) strain is 1:9. Preferably, the stirrer rotation speed remains at 244 to 344 rpm.
(4) No regulation of the pH value is conducted. The biomass is processed according to methods generally known to persons skilled in the art, such as using solid-fluid separation or extraction of the solid material, if necessary following gentle drying of the biomass pellet.

In summary, it can be determined that the present invention guarantees high productivity of Blakeslea strains through the control of physical parameters alone, as a result of which no problems can be anticipated with regard to the authorisation of the carotenoids produced for use as foodstuffs or for pharmaceutical purposes, since due to the method according to the invention, no toxic or otherwise hazardous substances are left behind in the product. With the method according to the invention, carotenoid yields in the biological dry mass of at least 2% can be obtained, preferably from 4 to 6%.

With the method according to the invention, the carotenoid can not only be extracted from the biomass obtained, but as an alternative or additionally can also be extracted from the oil phase of the fermentation sludge.

It is known that in Blakeslee trispora, the carotenoid formation increasingly occurs during the idiophase, wherein initially in the (−) strain a lipid or oil interstratification occurs. The carotenoid, in particular the hydrophobic β-carotene, is interstratified into this intracellular oil phase. Following completion of the fermentation, the carotenoid can be extracted from the biomass obtained, whereby the cells are decomposed and the carotenoid or carotenoid-oil mixture, is extracted from the intracellular oil phase.

It has surprisingly been shown that with the design of the method according to the invention, significant quantities of carotenoid are released by the organisms during fermentation and discharged into the fermentation sludge as a carotenoid-oil mixture. This permits the method according to the invention to extract carotenoid using separation of the oil phase of the fermentation sludge. No cell decomposition is necessary and the carotenoid extracted can be further used without further process steps.

The method according to the invention for producing a carotenoid selected from β-carotene or lycopene through submerged fermentation with mixed cultures of (+) and (−) strains of the fungus Blakeslee trispora and the extraction of the carotenoid from the oil phase of the fermentation sludge is characterized by the fact that initially, the (−) and the (+) strain are separately pre-cultivated up to full morphological formation, then the (−) strain is inoculated in the bioreactor, and during the exponential growth of the (−) strain, the (+) strain is added to the (−) strain in the volume ratio 1:5 to 1:100 (also known as the mating ratio) for the induction of the carotenoid formation, and both strains are jointly fermented, without pH regulation and without regulation of the partial oxygen pressure, at 18 to 24° C., if necessary in the phase of carotenoid formation (idiophase). Then, the oil phase is separated from the fermentation sludge and if necessary filtered, so that macroscopic components, i.e. components with an average diameter of 1 μm or more, are removed from the oil phase. As a result, oil is obtained which contains carotenoid, which as such can be further used, or which can be subjected to further purification in order to obtain even purer carotenoid fractions.

Due to the fact that the organisms release carotenoid formed during the idiophase as a carotenoid-oil mixture from the inside of the cell, in the method according to the invention, an oil phase is created in the fermentation sludge from which the carotenoid can be extracted, regardless of whether or not the fermentation substrate itself contains a lipid or oil.

Preferably, in the method according to the invention, substrates from the fermentation sludge are used to extract the carotenoid which contains one or more lipids. The lipids are preferably one or more oils. The oils are preferably one or more native oils. In particular, vegetable oils, preferably native vegetable oils, can be used in the substrate. Examples of suitable vegetable oils are olive oil, rapeseed oil, maize germ oil and sunflower oil.

In contrast to methods used to date, in which the carotenoid is extracted from the obtained biomass, and which are thus usually designed to be discontinuous, the method according to the invention also permits a continuous or at least multi-cyclical method implementation in order to extract carotenoids from the oil phase of the fermentation sludge. In order to guarantee a multi-cyclical or continuous method implementation, substrate is again added to the fermentation sediment after induction, so that the nutrient offer in the fermentation sediment permits a further culture of the sediment. The addition of fermentation substrate can here be conducted once, multiple times or at regular intervals.

Preferably, fermentation substrate is added in such a manner that in the fermentation sediment, the concentration of the carbon source does not fall below a threshold value at which the organisms die off. For example, the first addition of fermentation substrate occurs during a time period of 20 h to 60 h following induction by adding the (+) strain.

The fermentation substrate can be added multiple times or at regular intervals. For example, the first addition of the fermentation substrate occurs during a time period of 20 h to 60 h following induction by adding the (+) strain, while every further addition is made within a time period of 24 h to 48 h after the previous addition. The timespan between each further addition can also be shorter or longer. A decisive factor is that for the duration or for a longer period of time, the substrate content does not decrease below a concentration which leads to inhibition of the fungus.

In a particular embodiment, the method according to the invention for producing a carotenoid selected from β-carotene or lycopene through submerged fermentation with mixed cultures of (+) and (−) strains of the fungus Blakeslee trispora and the extraction of the carotenoid from the oil phase of the fermentation sludge is characterized by the following steps:

(1) The (−) strain is pre-cultivated separately for approx. 55 h and the (+) strain is pre-cultivated separately for approx. 68 h in the erlenmeyer flask at 28° C. and 145 rpm.

(2) After approx. 55 h, the (−) strain is transferred to a reactor and fermented for approx. 13-14 h at 28° C., a fumigation rate of approx. 1 vvm and a stirrer speed of 244 to 344 rpm.

(3) Following induction of the carotenoid production through the addition of the (+) strain, the cultivation temperature is set to 22° C. The volume ratio between the (+) and (−) strain is 1:9.

(4) No regulation of the pH value is conducted.

(5) After 20 h-60 h following addition of the (+) strain, a new fermentation substrate, as an option with oil, is added to the fermentation sludge.

(6) As an option, further additions of fermentation substrate are made to the fermentation sludge, wherein each further addition is made within a time period of 24 h to 48 h after the previous addition of fermentation substrate. In the interval, the fermentation is preferably continued in unchanged conditions.

(7) Separation of the oil phase (containing carotenoid) from the remaining fermentation sludge and if necessary, filtration of the oil or oil mixture containing carotenoid extracted.

In the method according to the invention, e.g. due to altered ambient conditions such as the renewed or further feed of nutrients through the addition of fermentation substrate, the secondary digestion products such as carotenoids, are excreted in microbial oil from the fungi. This process is similar to a detoxification and secures the continued existence of the fungus. Due to the sluicing out of the intracellular carotenoid-oil mixture into the fermentation sludge, the carotenoid can directly be extracted as an oil phase from the fermentation sludge, and a high-cost, time-consuming separation of the carotenoids from the biomass is no longer required. As a result, no mechanical, thermal, enzymatic or chemical treatment of the biomass is any longer necessary; the carotenoids can be removed as native carotenoids directly as an oil phase of the fermentation sludge. Due to the method implementation recommended here, the extraction of carotenoids can be conducted as a continuous method, in which higher yields occur than with the convention method implementation, in particular when the carotenoids are not only extracted from the oil phase of the fermentation sludge, but also from the biomass.

Through multiple follow-up feeding with fermentation substrate, in the method according to the invention, the content of carotenoids and xanthophylls can be increased in the oil product.

In order to permit a continuous or cyclical method implementation, with as many follow-up feeding cycles as possible, in the method according to the invention, the one-off or multiple further addition of (+) or (−) hemi-strain can be made and accordingly, the oil can continue to be enriched with carotenoids.

It has been shown that by means of the method according to the invention, in particular when extracting carotenoids from the oil phase of the fermentation sludge, oils or oil mixtures can be produced which have a carotenoid content, in particular a native carotenoid content, of 400 mg/l or more, preferably of 1,500 mg/l or more, and in a particularly preferred manner, of 400 to 1,500 mg/l and more.

It has been shown that by means of the method according to the invention, in particular when extracting carotenoids from the oil phase of the fermentation sludge, oils or oils mixtures can be produced which contain lycopene, in particular lycopene, at a level of 2 mg/l, preferably of 2 to 20 mg/l and more.

The method according to the invention therefore permits the production of native carotenoid which has not been impaired by processes during cell decomposition. Thus, the present invention also relates to native carotenoid or to an oil or oil mixture containing a native carotenoid, wherein the native carotenoid can be produced or is produced according to a method according to the invention. The oil or oil mixture containing carotenoid according to the invention preferably comprises a concentration of native carotenoid of 400 mg/l or more.

The oil or oil mixture containing carotenoid according to the invention is preferably characterized in that the concentration of β-carotene is 20 to 1,500 mg/l, particularly preferred, 30 to 1,300 mg/l, and very particularly preferred 300 to 1,300 mg/l.

The oil or oil mixture containing carotenoid produced using the method according to the invention preferably comprises a concentration of lycopene of more than 2 mg/l, particularly preferred from 2 to 29 mg/l, very particularly preferred from 2 to 15 mg/l.

In a particular embodiment, the oil or oil mixture containing carotenoid comprises at least 30 mg/l β-carotene and at least 2 mg/l lycopene, preferably at least 400 mg/l β-carotene and at least 5 mg/l lycopene.

The oil or oil mixture containing carotenoid can for example contain:

≧0.25 mg/l lutein,

≧2 mg/l β-cryptoxanthine,

≧2 mg/l lycopene,

≧3 mg/l gamma-carotene,

≧0.25 mg/l alpha-carotene, and

≧30 mg/l β-carotene.

For example, the oil or oil mixture containing carotenoid can contain:

0.25 to 1 mg/l lutein,

2 to 25 mg/l β-cryptoxanthine,

2 to 15 mg/l lycopene,

3 to 35 mg/l gamma-carotene,

0.2 to 25 mg/l alpha-carotene, and

30 to 1300 mg/l β-carotene.

In particular, the oil or oil mixture containing carotenoid can for example contain:

0.28 to 0.71 mg/l lutein,

2.05 to 24.9 mg/l β-cryptoxanthine,

2.09 to 14.35 mg/l lycopene,

3.01 to 31.8 mg/l gamma-carotene,

0.29 to 24.9 mg/l alpha-carotene, and

30.9 to 1295 mg/l β-carotene.

The invention will now be explained in greater detail below with reference to examples, although without restricting it to these.

Figures:

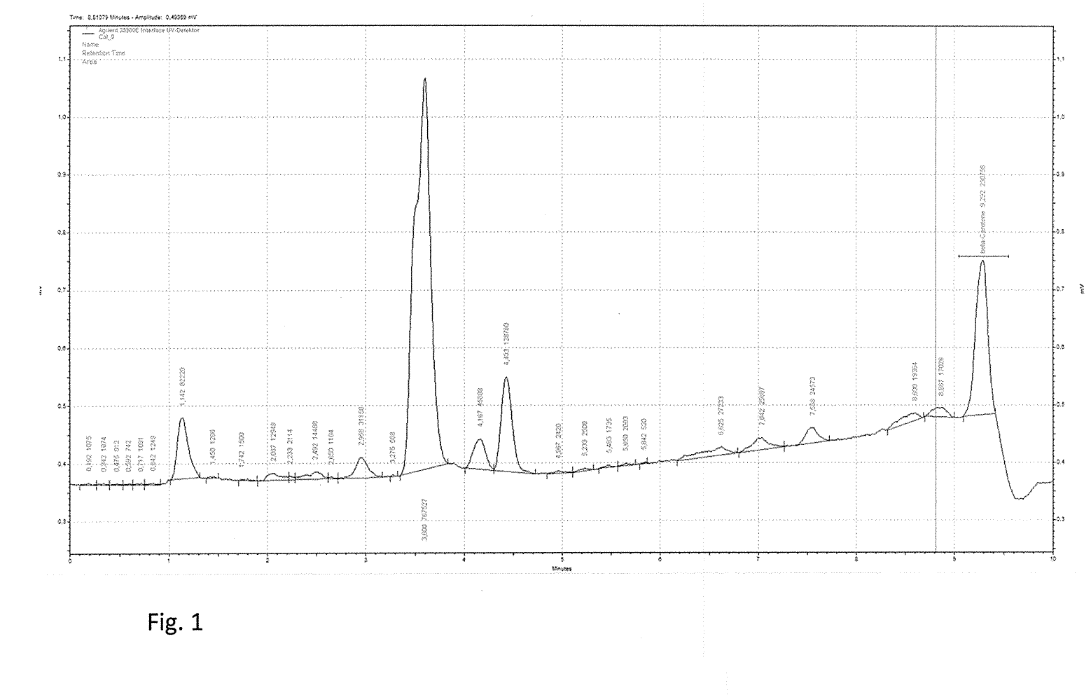

In FIGS. 1 to 7:

FIG. 1: shows and HPLC chromatogram of the native sunflower oil.

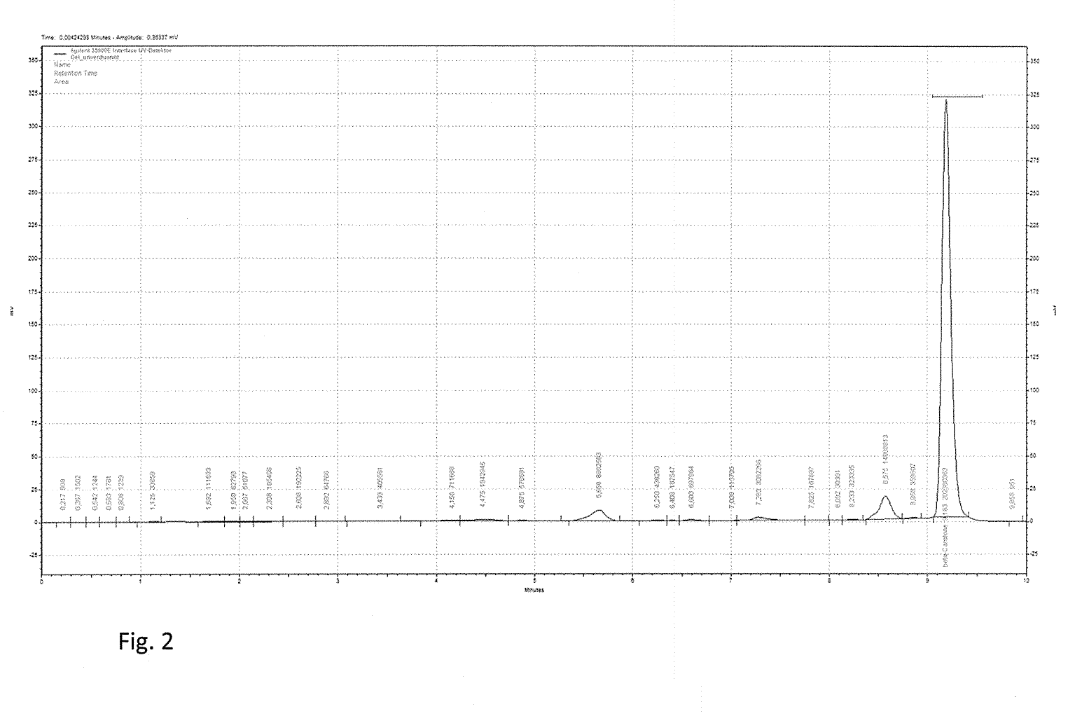

FIG. 2: shows an HPLC chromatogram of the oil containing carotenoid after 3 subsequent feeds with substrate containing starch (sunflower oil).

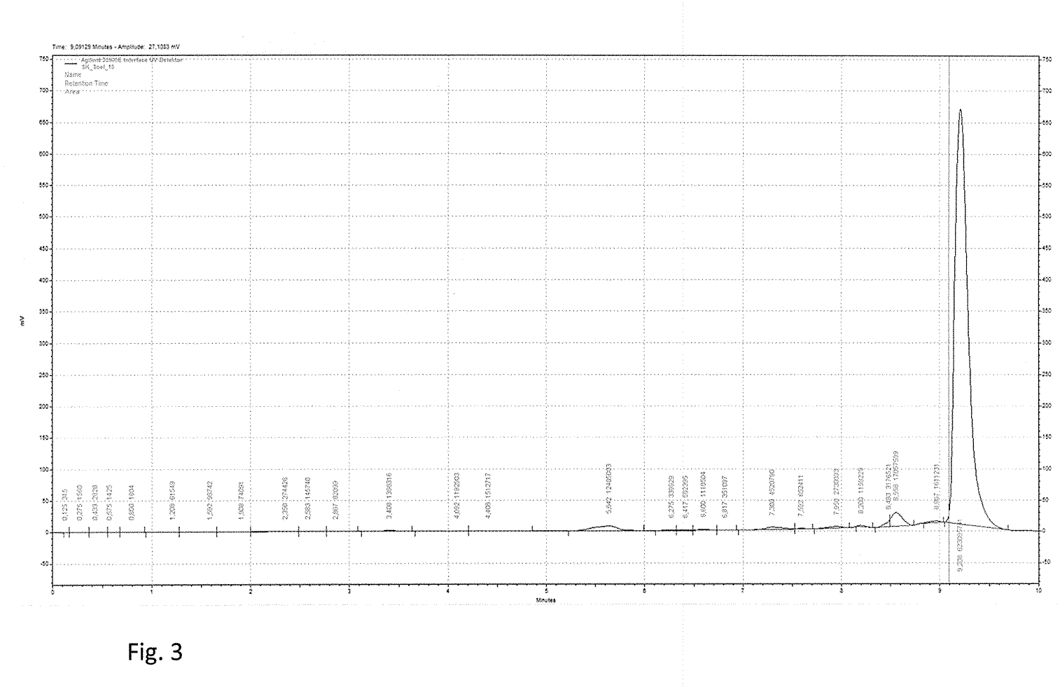

FIG. 3: shows an HPLC chromatogram of the oil containing carotenoid after 5 subsequent feeds with substrate containing starch (sunflower oil).

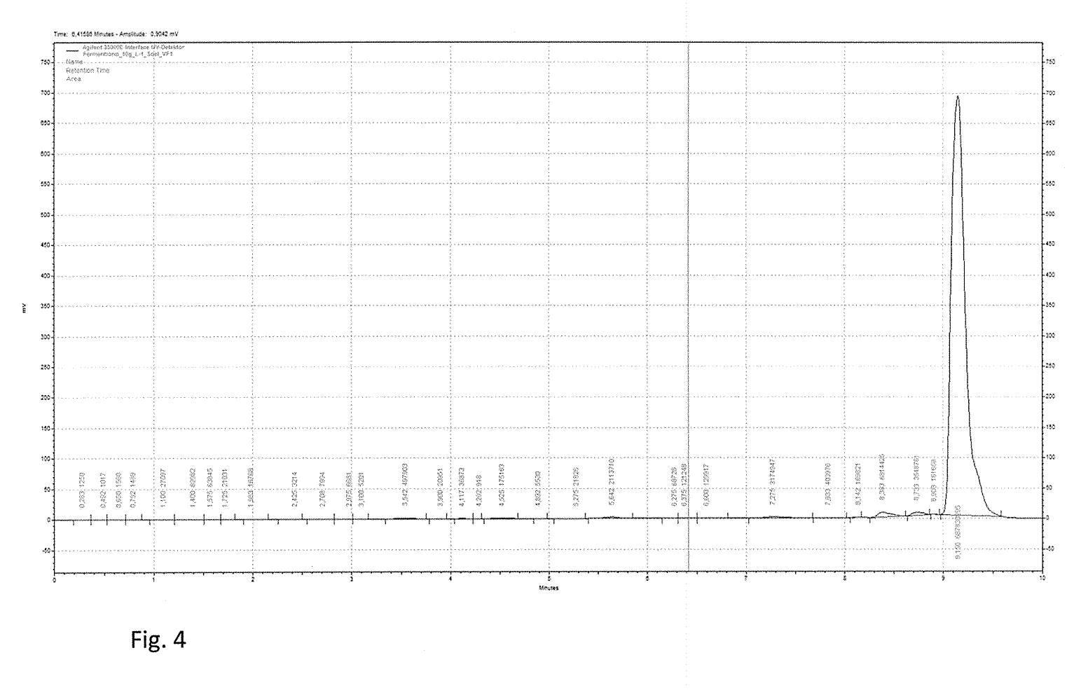

FIG. 4: shows an HPLC chromatogram of the oil containing carotenoid after 5 subsequent feeds with substrate containing glucose (sunflower oil).

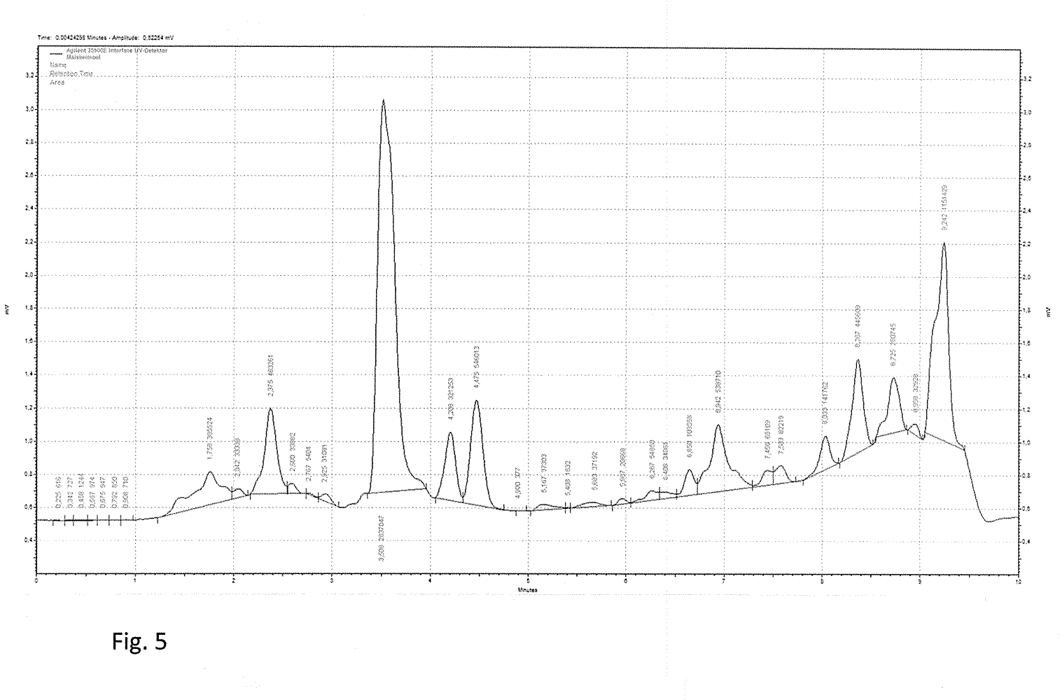

FIG. 5: shows an HPLC chromatogram of the maize germ oil.

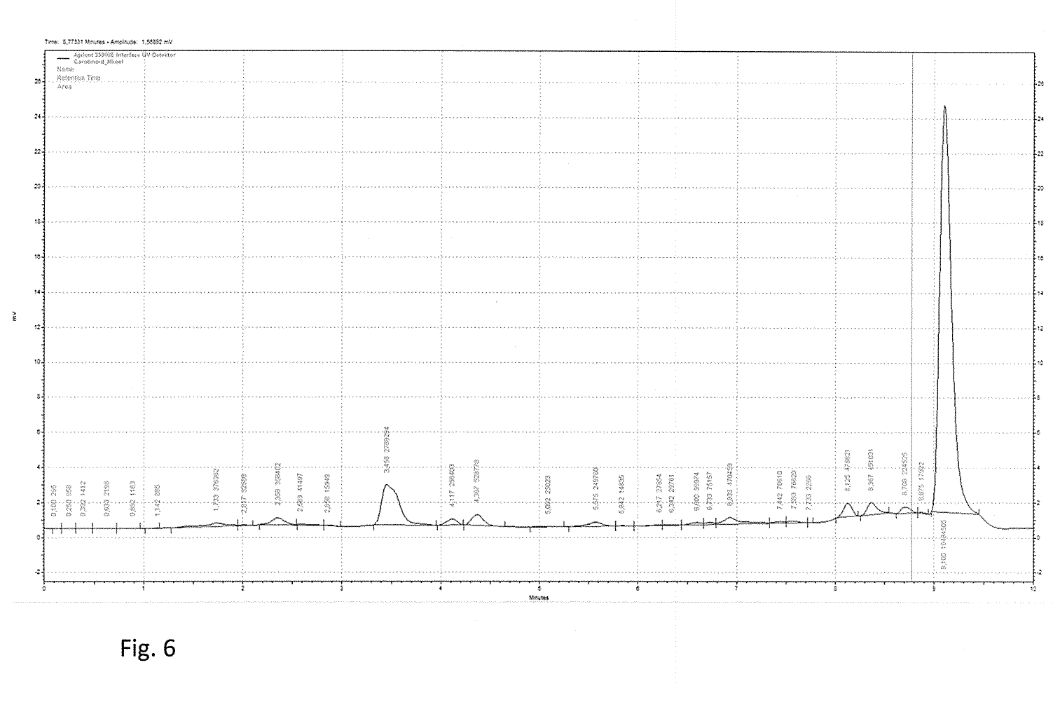

FIG. 6: shows an HPLC chromatogram of the oil containing carotenoid after one subsequent feed with substrate containing start (maize germ oil).

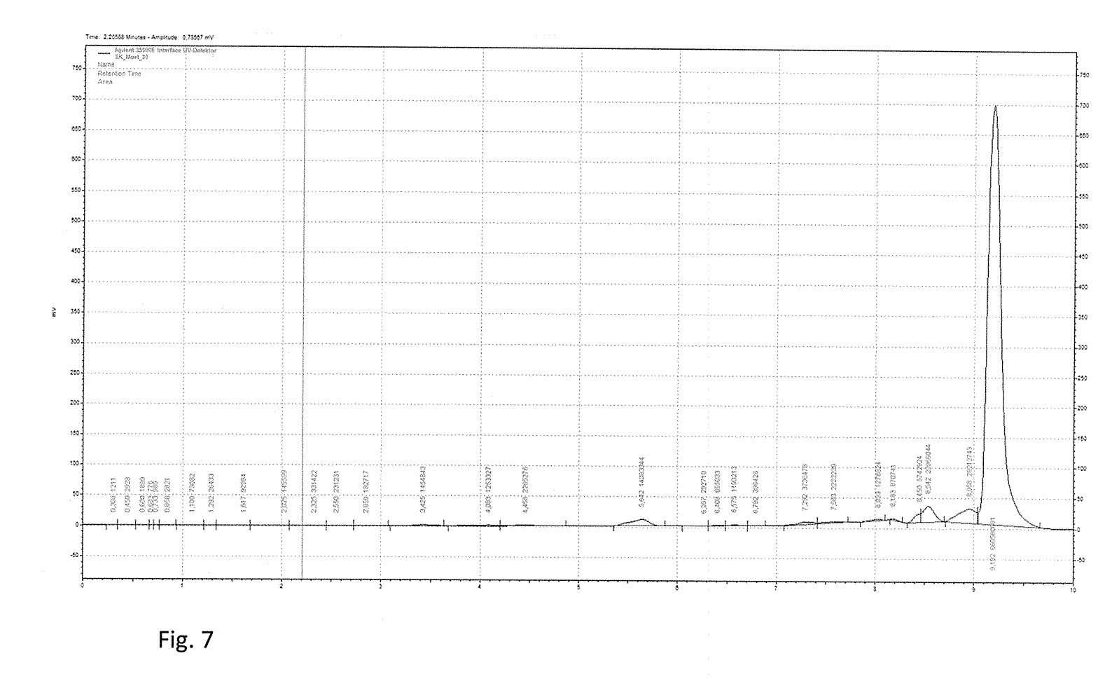

FIG. 7: shows an HPLC chromatogram of the oil containing carotenoid after 5 subsequent feeds with substrate containing starch (maize germ oil).

### EXAMPLES

### Example 1

The hemi-strains DSM 2387 and DSM 2388 are separately cultivated on YPsS solid medium (starch: 15 g/l, yeast extract: 4 g/l, magnesium sulphate: 0.45 g/l, dipotassium hydrogen phosphate: 1.0 g/l, agar: 20 g/l). The formation of spores begins within 3 days. From the 3rd day of cultivation, the spores can be rinsed off sterile with a 0.2% Span20 solution.

A 300 ml flask is filled with 200 ml YPsS medium (starch: 15 g/l, yeast extract: 4 g/l, magnesium sulphate: 0.45 g/l, dipotassium hydrogen phosphate: 1.0 g/l, agar: 20 g/l, thiamine: 0.002 g/l, sunflower oil: 30 g/l). The medium, which is steam-sterilised at 121° C. for 20 minutes, is injected with 2*10⁶ spores after cooling to room temperature. The (−) strain is pre-cultivated for 55 hours and the (+) strain for 68 hours, separately from each other at 28° C. The shaking frequency is 145 rpm.

A reactor with a nominal volume of 5 l is filled with 3200 ml of the fluid medium (YPsS without yeast extract) and sterilised for 30 minutes at 121° C. After cooling of the medium, the sterile yeast extract (360 ml) and thiamine (12 ml) are added. The portion of the individual components of the fluid medium was weighed at 3572 ml. After setting the cultivation temperature at 28° C., the reactor is injected with 200 ml of the DSM 2388 culture. A cultivation occurs for 13 hours, so that the fungus is within the exponential growth phase. Then, 400 ml of the DSM 2387 culture is added for induction of the carotenoid formation. For the idiophase, the temperature is reduced to 22° C. An intensive orange coloration of the biomass begins within the 36 to 48 hours.

For cultivation, neither a control of the pH value nor of the pO₂ value is required. Fumigation is conducted throughout with 1 vvm at a stirrer speed of 300 rpm.

Following completion of the cultivation, the biomass is processed (solid-fluid separation, extraction of the solid substance, if required, after gentle drying of the biomass pellet). The yield of β-carotene in the biomass is 3.2 to 6.18%.

### Example 2

The hemi-strains DSM 2387 and DSM 2388 are separately cultivated on YPsS solid medium (starch: 15 g/l, yeast extract: 4 g/l, magnesium sulphate: 0.45 g/l, dipotassium hydrogen phosphate: 1.0 g/l, agar: 20 g/l). The formation of spores begins within 3 days. From the 3rd day of cultivation, the spores can be rinsed off sterile with a 0.2% Span20 solution.

A 300 ml flask is filled with 200 ml YPsS medium (beer wort: 10 g/l, yeast extract: 4 g/l, magnesium sulphate: 0.45 g/l, dipotassium hydrogen phosphate: 1.0 g/l, agar: 2 g/l, thiamine: 0.002 g/l, sunflower oil: 30 g/l). The medium, which is steam-sterilised at 121° C. for 20 minutes, is injected with 2*10⁶ spores after cooling to room temperature. The (−) strain is pre-cultivated for 55 hours and the (+) strain for 68 hours, separately from each other at 28° C. The shaking frequency is 145 rpm.

A reactor with a nominal volume of 5 l is filled with 3200 ml of the fluid (beer wort: 10 g/l, magnesium sulphate: 0.45 g/l, dipotassium hydrogen phosphate: 1.0 g/l, agar: 2 g/l, sunflower oil: 30 g/l) and sterilised for 30 minutes at 121° C. After cooling of the medium, the sterile yeast extract (14.292 g/360 ml) and thiamine (0.0072 g/12 ml) are added. The portion of the individual components of the fluid medium was weighed at 3572 ml. After setting the cultivation temperature at 28° C., the reactor is injected with 200 ml of the DSM 2388 culture. A cultivation occurs for 13 hours, so that the fungus is within the exponential growth phase. Then, 400 ml of the DSM 2387 culture is added for induction of the carotenoid formation. For the idiophase, the temperature is reduced to 22° C. An intensive orange coloration of the biomass begins within the 36 to 48 hours.

For cultivation, neither a control of the pH value nor of the p02 value is required. Fumigation is conducted throughout with 1 vvm at a stirrer speed of 300 rpm.

Following completion of the cultivation, the biomass is processed (solid-fluid separation, extraction of the solid substance, if required, after gentle drying of the biomass pellet). The yield of β-carotene in the biomass is 1.8 to 2.3%.

### Example 3

The hemi-strains ATCC 14271 and ATCC 14272 are separately cultivated on YPsS solid medium (starch: 15 g/l, yeast extract: 4 g/l, magnesium sulphate: 0.45 g/l, dipotassium hydrogen phosphate: 1.0 g/l, agar: 20 g/l). The formation of spores begins within 3 days. From the 3rd day of cultivation, the spores can be rinsed off sterile with a 0.2% Span20 solution.

A 300 ml flask is filled with 200 ml YPsS medium (starch: 15 g/l, yeast extract: 4 g/l, magnesium sulphate: 0.45 g/l, dipotassium hydrogen phosphate: 1.0 g/l, agar: 2 g/l, thiamine: 0.002 g/l, sunflower oil: 30 g/1). The medium, which is steam-sterilised at 121° C. for 20 minutes, is injected with 2*10⁶ spores after cooling to room temperature. The (−) strain is pre-cultivated for 55 hours and the (+) strain for 68 hours, separately from each other at 28° C. The shaking frequency is 145 rpm.

Two reactors with a nominal volume of 5 I are filled with 3200 ml of the fluid (YPsS without yeast extract) and sterilised for 30 minutes at 121° C. After cooling of the medium, the sterile yeast extract (360 ml) and thiamine (12 ml) are added. The portion of the individual components of the fluid medium was weighed at 3572 ml. After setting the cultivation temperature at 28° C., the reactors are injected with 200 ml of the ATCC 14721 culture. A cultivation occurs for 14 hours, so that the fungus is within the exponential growth phase.

In a reactor with a nominal volume of 15 I, 9 I YPsS medium are introduced, sterilized and injected with 500 ml of ATCC 14272. The fungus is in the exponential growth phase after 14 hours. 1 1 of the ATCC 14271 culture is added for the induction of the product formation. For the idiophase, the temperature is reduced to 22° C. Within the next 50 to 64 hours, an intensive orange coloration of the biomass begins.

For cultivation, neither a control of the ph value nor of the pO₂ value is required. Fumigation is conducted throughout with 1 vvm at a stirrer speed of 300 rpm.

Following completion of the cultivation, the biomass is processed (solid-fluid separation, extraction of the solid substance, if required, after gentle drying of the biomass pellet). The yield of β-carotene in the biomass is 3.8 to 4.5%.

### Example 4

The hemi-strains DSM 2387 and DSM 2388 are separately cultivated on YPsS solid medium (starch: 15 g/l, yeast extract: 4 g/l, magnesium sulphate: 0.45 g/l, dipotassium hydrogen phosphate: 1.0 g/l, agar: 20 g/l). The formation of spores begins within 3 days. From the 3rd day of cultivation, the spores can be rinsed off sterile with a 0.2% Span20 solution.

A 300 ml flask is filled with 200 ml YPsS medium (starch: 15 g/l, yeast extract: 4 g/l, magnesium sulphate: 0.45 g/l, dipotassium hydrogen phosphate: 1.0 g/l, agar: 2 g/l, thiamine: 0.002 g/l, sunflower oil: 30 g/l). The medium, which is steam-sterilised at 121° C. for 20 minutes, is injected with 2*10⁶ spores after cooling to room temperature. The (−) strain is pre-cultivated for 55 hours and the (+) strain for 68 hours, separately from each other at 28° C. The shaking frequency is 145 rpm.

A reactor with a nominal volume of 5 l is filled with 3200 ml of the fluid (YPsS without yeast extract) and sterilised for 30 minutes at 121° C. After cooling of the medium, the sterile yeast extract (360 ml) and thiamine (12 ml) are added. The portion of the individual components of the fluid medium was weighed at 3572 ml. After setting the cultivation temperature at 28° C., the reactor is injected with 200 ml of the DSM 2387 culture. A cultivation occurs for 13 hours, so that the fungus is within the exponential growth phase. Then, 400 ml of the DSM 2387 culture is added for induction of the carotenoid formation. For the idiophase, the temperature is reduced to 22° C. An intensive orange coloration of the biomass begins within the 36 to 48 hours.

For cultivation, neither a control of the pH value nor of the pO₂ value is required. Fumigation is conducted throughout with 1 vvm at a stirrer speed of 300 rpm.

After 24h following addition of the (+) strain, new fermentation substrate is added to the fermentation sludge. The addition of cultivation medium is repeated several times at an interval of 12 to 48 hours, thus intensifying the effect. Within 10 to 48 hours, an intensive orange to red coloration of the sunflower oil begins. Following completion of the cultivation, the biomass and the oil phase is processed (solid-fluid separation, extraction of the solid substance, if required, after gentle drying of the biomass pellet). The yield of β-carotene in the biomass is 3 to 6%, the content of β-carotene in the oil phase is 500 to 1500 mg I-1 with new fermentation substrate added 7 times.

### Example 5

The hemi-strains ATCC 14271 and ATCC 14272 are separately cultivated on YPsS solid medium (starch: 15 g/l, yeast extract: 4 g/l, magnesium sulphate: 0.45 g/l, dipotassium hydrogen phosphate: 1.0 g/l, agar: 20 g/l). The formation of spores begins within 3 days. From the 3rd day of cultivation, the spores can be rinsed off sterile with a 0.2% Span20 solution.

A 300 ml flask is filled with 200 ml YPsS medium (starch: 15 g/l, yeast extract: 4 g/l, magnesium sulphate: 0.45 g/l, dipotassium hydrogen phosphate: 1.0 g/l, agar: 2 g/l, thiamine: 0.002 g/l, sunflower oil: 30 g/l). The medium, which is steam-sterilised at 121° C. for 20 minutes, is injected with 2*10⁶ spores after cooling to room temperature. The (−) strain is pre-cultivated for 55 hours and the (+) strain for 68 hours, separately from each other at 28° C. The shaking frequency is 145 rpm.

A reactor with a nominal volume of 5 I is filled with 3200 ml of the fluid (YPsS without yeast extract) and sterilised for 30 minutes at 121° C. After cooling of the medium, the sterile yeast extract (360 ml) and thiamine (12 ml) are added. The portion of the individual components of the fluid medium was weighed at 3572 ml. After setting the cultivation temperature at 28° C., the reactor is injected with 200 ml of the DSM 2387 culture. A cultivation occurs for 13 hours, so that the fungus is within the exponential growth phase. Then, 400 ml of the DSM 2387 culture is added for induction of the carotenoid formation. For the idiophase, the temperature is reduced to 22° C. An intensive orange coloration of the biomass begins within the 36 to 48 hours.

For cultivation, neither a control of the pH value nor of the pO₂ value is required. Fumigation is conducted throughout with 1 vvm at a stirrer speed of 300 rpm.

After 30h following addition of the (+) strain, new fermentation substrate is added to the fermentation sludge. The addition of cultivation medium is repeated several times at an interval of 12 to 48 hours, thus intensifying the effect. Within 10 to 48 hours, an intensive orange to red coloration of the sunflower oil begins. Following completion of the cultivation, the biomass and the oil phase is processed (solid-fluid separation, extraction of the solid substance, if required, after gentle drying of the biomass pellet). The yield of β-carotene in the biomass is 3 to 6%, the content of β-carotene in the oil phase is 400 to 600 mg I-1 with new fermentation substrate added 4 times.

### Example 6

In order to determine the oil composition with respect to the carotenoids and xanthophylls, an HPLC analysis (Table 1) of the oils containing carotenoid is conducted of examples 4 and 5.

TABLE 1
HPLC methods

| t [min] | A (Acetone) [%] | B (Water) [%] |
|---|---|---|
| 0.0 | 80 | 20 |
| 7.0 | 100 | 0 |
| 7.5 | 80 | 20 |
| 12 | 80 | 20 |

T = 30° C.;
Vᵢₙⱼ. = 10 μL;
Column: C18 (Agilent XDB);
Detection: λ= 450 nm;
Flow: 1 ^{mL}/ₘᵢₙ

HPLC:

Pump: Merck Hitachi L-6200A

Autosampler: Merck Hitachi AS-4000A

UV-VIS detector: Merck Hitachi L-4250

Interface: Agilent Interface 35900E

Software: OpenLAB

In order to determine the concentration of carotenoids and xanthophylls in the oil according to the invention, strain solutions of the existing standards (table 2) are produced. For this purpose, a defined quantity of oil or acetone (Carl Roth GmbH+Co. KG) is dissolved and stored in a brown bottle. Before the HPLC analysis, the strain solutions were filtered in order to guarantee a particle-free solution. On the basis of the calibration degree determined (concentration to area), conclusions could be reached regarding the concentration from the peak area in the oil containing carotenoid.

TABLE 2
Standards

| | | S_{train solution} |
|---|---|---|
| Standard | Supplier | [mg l⁻¹] |
| Zeaxanthine | Sigma Aldrich | |
| Lutein | Sigma Aldrich | 45 |
| β-cryptoxanthene | Sigma Aldrich | 12.8 |
| Lycopene | VWR | 20 |
| γ-carotene | CaroteNature | 10 |
| α-carotene | Sigma Aldrich | 20 |
| β-carotene | Sigma Aldrich | 130 |

In order to analyse the oils containing carotenoid, the oil, although also parts of the fermentation sludge and biomass, was separated by decanting (pouring out). Through centrifugation of the sample at 3044 g for 5 mins., a clear separation between the biomass, fermentation sludge and oil phase can be made. The oil samples were removed and filtered for the HPLC determination (pore size 0.45 μm; PTFE injection filter; Carl Roth GmbH+Co. KG), in order to guarantee a particle-free sample. This approach is particularly suited to the laboratory scale; for implementation on the technical scale, separators and decanters should be used for fluid-fluid separation.

Sample spectres of the oils studied are shown in FIGS. 1 to 7. Here, FIG. 1 shows an HPLC chromatogram of the native sunflower oil; FIG. 2 shows an HPLC chromatogram of an oil containing carotenoid according to example 4 after 3 subsequent feeds with substrate containing starch (sunflower oil); FIG. 3 shows an HPLC chromatogram of an oil containing carotenoid according to example 4 after 5 subsequent feeds with substrate containing starch (sunflower oil); FIG. 4 shows an HPLC chromatogram of an oil containing carotenoid according to example 4 after 5 subsequent feeds with substrate containing glucose (sunflower oil); FIG. 5 shows an HPLC chromatogram of the maize germ oil; FIG. 6 shows an HPLC chromatogram of an oil containing carotenoid according to example 5 following a subsequent feed with a substrate containing starch (maize germ oil); and FIG. 7 shows an HPLC chromatogram of an oil containing carotenoid according to example 5 after 5 subsequent feeds with substrate containing starch (maize germ oil).

In order to be able to precisely quantify the carotenoids and xanthophylls, the composition of the oils was determined in the first step. The peak areas calculated were subtracted from the peak areas of the oils containing carotenoids. The emerging significant peaks were detected as lutein, β-cryptoxanthene, lycopene, and α-, β- and γ-carotene (Table 3).

TABLE 3
Minima and maxima of the content determined via HPLC in mg l⁻¹ under
different cultivation conditions.

| | |
|---|---|
| MIN | MAX |

[mg l⁻¹]

| | | |
|---|---|---|
| Lutein | 0.28 | 0.71 |
| β- | 2.05 | 24.9 |
| cryptoxanthene | | |
| Lycopene | 2.09 | 14.35 |
| γ-carotene | 3.01 | 31.8 |
| α-carotene | 0.29 | 24.9 |
| β-carotene | 30.9 | 1295 |

The minima and maxima values here represent the lowest and highest values respectively of all oil samples containing carotenoid. The β-carotenoid content of 1295 mg l⁻¹ was achieved on the reactor scale after 8 subsequent feeds. In the shake flask attempts, the maximum was around 700 mg l⁻¹ after approx. 4 to 5 subsequent feeds.

The content of carotenoids in the oil phase was dependent on the method strategy. After one to two subsequent feeds, the content of e.g. β-carotene was approx. 50 to 100 mg l⁻¹. Through repeated subsequent feeds with a medium containing starch, molasses or glucose, the content of β-carotene in the oil could be increased, with four to five subsequent feeds to approx. 500 to 800 mg l⁻¹ and with 7 to 10 subsequent feeds, to over 1000 mg l⁻. The interval between feeds was in the present studies between 20 and 50 hours, although a shorter or longer interval is also possible. The content of carotenoids and xanthophylls primarily depends on the number of subsequent feeds, the oil used and the substrate.

The main portion of carotenoids contained approx. 80 to 97% β-carotene, with detection of the other carotenoids dependent of the substrate used, the oil used and the number of subsequent feeds. In particular, the number of subsequent feeds lowers the portion of other carotenoids and thus increases the percentage portion of β-carotene, wherein through synthesis processes, a change to the content in the oil containing carotenoid can also occur.

1. A method for producing a carotenoid comprising one or more of β-carotene and lycopene through submerged fermentation with mixed cultures of (+) and (−) strains of the fungus Blakeslea trispora and extracting the carotenoid from one or more of an obtained biomass and an oil phase of a fermentation sludge, the method comprising:
separately pre-cultivating, the (−) and the (+) strains of Blakeslea trispora a full morphological formation;
inoculating and cultivating the (−) strain in a bioreactor;
during the exponential growth of the (−) strain in the bioreactor, adding the (+) strain to the bioreactor in a volume ratio of 1:5 to 1:100 to induce carotenoid formation; and
jointly fermenting the (−) and the (+) strains in the bioreactor a temperature between 18 to 24° C., the bioreactor being free from pH regulation and free from regulation of the oxygen partial pressure.

2. The method according to claim 1, wherein both the (−) and the (+) strains are fermented jointly in the bioreactor until carotenoid formation has been completed.

3. The method according to claim 1 or, wherein the method steps of separately pre-cultivating the (−) and the (+) strains and inoculating and cultivating the (−) strain in the bioreactor are conducted at a temperature between 26-30° C.

4. The method according to claim 1, wherein the (+) strain is added to the (−) strain at a volume ratio of 1:5 to 1:20.

5. The method according to claim 1, wherein the (+) and (−) strains are jointly fermented for carotenoid formation at a temperature of 22° C.

6. The method according to claim 1, wherein a substrate for the joint fermentation of the (+) and (−) strains comprises one or more carbohydrates and one or more organic nitrogen compounds.

7. The method according to claim 6, wherein the one or more carbohydrates are selected from the group consisting of: starch, molasses for beer wort, and combinations thereof.

8. The method according to claim 1, wherein a substrate for the joint fermentation of the (+) and (−) strains comprises lipids.

9. The method according to claim 1, wherein the carotenoid comprises lycopene and a cyclase inhibitor is added to a substrate for the fermentation of the (+) and (−) strain.

10. The method according to claim 1, wherein a fumigation rate, a stirrer speed, and a temperature of the bioreactor are kept constant during joint fermentation of the (−) and the (+) strains.

11. The method according to claim 1, wherein the (−) strain comprises DSM 2388 and the (+) strain comprises DSM 2387 or the (−) strain comprises ATCC 14272 and the (+) strain comprises ATCC 14271.

12. The method according to claim 1, wherein the oil phase containing the carotenoid is separated out from the remaining fermentation sludge.

13. The method according to claim 12, wherein the oil phase containing the carotenoid is filtered.

14. The method according to claim 1, wherein the method is continuously operated.

15. The method according to claim 1, wherein a substrate is added anew to a fermentation sediment after induction.

16. The method according to claim 15, wherein addition of the substrate occurs in a period of 20 h to 60 h following induction of the fermentation sediment through adding the (+) strain.

17. The method according to claim 16, wherein further additions of the substrate are made, wherein each of the further additions is made within a period of 24 h to 48 h after the addition of a most recently added substrate.

18. An oil or oil mixture containing native carotenoid, wherein that the oil or oil mixture is produced using a method according to claim 1.

19. The oil or oil mixture according to claim 18, wherein the oil or oil mixture comprises a concentration of native carotenoid of 400 mg/l or more.

20. The oil or oil mixture according to claim 18, wherein the oil or oil mixture comprises a concentration of at least 400 mg/l β-carotene and at least 5 mg/l lycopene.

21. The oil or oil mixture according to claim 18, wherein the oil or oil mixture comprises:
≧0.25 mg/l lutein,
≧2 mg/l β-cryptoxanthene,
≧2 mg/l lycopene,
≧3 mg/l gamma-carotene,
≧0.25 mg/l alpha-carotene, and
≧30 mg/l β-carotene.
